(19) United States
(12) Patent Application Publication
(10) Pub. No.: US 2010/0236547 A1
(43) Pub. Date: Sep. 23, 2010

(54) Container for aerosol drug delivery

(76) Inventor: Robert Owen Cook, Hillsborough, NJ (US)

Correspondence Address: Owen J. Bates, Suite 200, 2400 Bayshore Parkway, Mountain View, CA, 94043, US

(21) Appl. No.: 12/459,954

(22) Filed: Jul. 10, 2009

Related U.S. Application Data
(60) Provisional application No. 61/080,213, filed on Jul. 11, 2008.

(51) Int. Cl. A61M 11/02

(52) U.S. Cl. 128/200.23

(57) ABSTRACT

A metered does inhaler which includes a canister assembly which has a minimal volume canister and primeless valve to reduce waste, minimize total drug contained therein, and increase the reproducibility of dosage delivered.

### CROSS-REFERENCE TO RELATED APPLICATIONS

This application claims the benefit of: U.S. Provisional Application No. 61/080,213, filed 11 Jul. 2008, entitled “Containers for Aerosol Drug Delivery” which is hereby incorporated by reference in its entirety.

### FIELD

Described here are metered dose inhalers including pressurized containers for delivering aerosolized active agents to the respiratory tract. Specifically, pressurized containers including valves lacking or having minimal priming requirements are described. Valves capable of reproducing unit doses of active agents upon minimal or no priming are also described.

### BACKGROUND

One of the key advantages of pressurized metered dose inhalers (pMDIs) is that they contain a reservoir of a solution or suspension of the drug in a propellant, in a canister sealed with a metering valve, the combination of which protects the drug from oxidation, moisture, light and other physicochemical degradation or contamination for extended storage periods, yet enables convenient, on demand and highly reproducible metering of fixed volumes—typically about 10 to about 200 microliters per actuation—of the drug/drug formulation. Conventional pMDIs include canisters that store enough drug for at least one month of typical patient usage, typically beta agonists or glucocorticosteroids for asthma treatments, and are adapted for dosing regimens of 1 to 8 actuations of the valve per day or 120 to 400 actuation volumes per canister. For existing typical formulations and drug dosages this regimen requires canisters that can hold between 5 and 19 milliliters of the drug formulation, including a small percentage of headspace, which is usually less than 20% of the canister volume.

For newer therapies, such as for the treatment of central nervous system ailments or pain, or for use with highly potent or expensive drugs, it is desirable to have the convenience and metering capability of the pMDI while minimizing the volume of the reservoir to conserve drug, minimize overage and hence reduce costs of goods. The amount of drug in pMDI reservoir should be minimized (total amount of drug supplied, number of doses or strength of preparations) for medicaments with narrow therapeutic windows, high potency, or medicaments that are restricted or controlled by government/regulatory bodies (controlled, listed or scheduled substances) to avoid unnecessary toxicity/overdose risk and or minimize abuse potential.

As the art is currently practiced, delivery of such newer therapeutics from a pMDI would comprise filling canisters with at least 5 milliliters of the formulation in canisters having a volume of at least 6 milliliters to 19 milliliters, and the use of retention valve technology (e.g., a Bespak 357 retention valve). With this current practice, a minimum of 70-80 doses are contained in these systems depending on the strength of the formulation and the valve size and thus represents a large overage of available doses in the canister. Aside from the potential to overdose because additional actuations significantly beyond the safe treatment amount can be administered repetitively, a canister with this volume of residual formulation may be pierced to extract a sufficient quantity of drug that could be used in abuse situations or serve as the starting material for the synthesis of other drugs.

Metering valves used with conventional pMDIs generally include a valve stem which is co-axially slidable within a valve member and defines an annular metering chamber. Outer and inner annular seals between the respective outer and inner ends of the valve stem and the valve member seal a metering chamber therebetween. The valve stem is movable between a non-dispensing position in which the metering chamber is connected to the container and charged with product therefrom. The valve stem is movable, usually against the biasing action of a spring, to a dispensing position where the metering chamber is isolated from the container and vented to the atmosphere which allows for the discharge of the product.

These conventional valves can lose prime when propellant is lost due to vapor or air getting trapped in the metering chamber, which replaces the active drug formulation. Loss of prime may also result from the tendency of the active agent to migrate out of the metering chamber during storage periods, particularly when the valves are stored with the valve in the upwards position. Thus, patients are generally advised to waste the first dose from devices having these types of valves, if the devices have not been used for a period of time. To make up for this waste, extra doses of product may be included in the containers. Loss of prime is commonly experienced with these conventional metering valves even in a period as short as 24 hours. A reduction in the delivery of as much as 75% of the product can occur between daily doses if priming before each delivery isn't performed. As previously mentioned, for drugs having a narrow therapeutic window or for controlled substances that may be restricted in the amounts supplied, number of doses, or strength of preparations, the presence of excess quantities to compensate for loss of prime increases the risk of toxicity, overdose, and/or drug abuse. Furthermore, even after being primed, the amount of product actually being delivered may be variable which is not desirable for a pharmaceutical product.]

Accordingly, containers that more effectively deliver doses of aerosolized product after periods of storage would be useful. In particular, valves capable of minimizing or eliminating loss of prime would be desirable. Valves that effectively deliver reproducible doses of active agents would also be desirable. Containers having minimal volume reservoirs that are just sufficient to enable the valve to have access to a metering volume for a sufficient number of actuations would be useful. Further, metered dose inhalers including such containers and valves to prevent overdose or drug abuse would also be useful.

### SUMMARY

The invention as described herein is a metered dose inhaler that generally includes a primeless valve and a minimal volume pressurized canister. As used herein, the term “primeless valve” refers to a non-retention valve. Exemplary non-retention valves are the Bespak 357 retention valve, or those valves described in U.S. Pat. No. 7,086,571, U.S. 2006/0231093, U.S. Pat. No. 7,040,513, and WO 08058539. The volume of the canisters or containers is usually smaller than those provided by conventional pressurized canisters/containers. In one variation, the minimal volume canister is a single, integral vessel. That is, the minimal or reduced volume is attributable to the size of the canister itself, and not to an insert or a second canister being disposed within the first canister.

In some variations of the invention, the pressurized canisters have a total volume of less than about 10 ml. In other variations, the pressurized canisters have a total volume of less than about 6.0 ml. In yet further variations, the pressurized canisters have a total volume of less than about 5.0 ml.

The pressuirze pressurized canisters of the invention may contain any active agent. For example, the active agent may be a central nervous system agent. It is understood that the terms “active agent”, “drug”, “product” and “medicament” are used interchangeably herein throughout. The may also be loaded with about 20 or fewer discharge volumes of active agent.

The pressurized canisters may be configured to have a ullage of less than about 0.9 ml. The may also be loaded with about 20 or fewer discharge volumes of active agent. In some variations, the primeless valves described here deliver a discharge volume of about 10 microliters to about 200 microliters.

### BRIEF DESCRIPTION OF THE DRAWINGS

FIG. 1 shows a side view of an exemplary valve and minimal volume canister.

### DETAILED DESCRIPTION

Described here are metered dose inhalers of the invention that generally include a primeless valve and a minimal volume pressurized canister. The combination of a primeless, i.e., non-retention valve, with a canister designed to provide the minimum volume around a valve to avoid unnecessary overfilling of drug formulation, drug loss to excessive surface areas of larger canisters and drug loss to priming shots is described. As previously mentioned, priming actuations are typically required and are fired to the environment with conventional valves, resulting in uncontrolled and unnecessary drug exposure and waste. By having a primeless valve, no priming is required and the first actuation of the canister/valve combination delivers a full dose. In some variations, reduced priming (in comparison to conventional valves) is employed.

In one variation of the invention, the primeless valve is capable of metering from 10 microliters to 200 microliters without recent previous priming, even after extended storage between usage, ranging from 1 day to about 2 years. The canister can be of diameters varying from about 8 millimeters to about 30 millimeters with heights varying between about 10 millimeters to about 30 millimeters. The canister may be formed from glass, plastic, metal, or combinations thereof, and coated or uncoated as appropriate for the formulation.

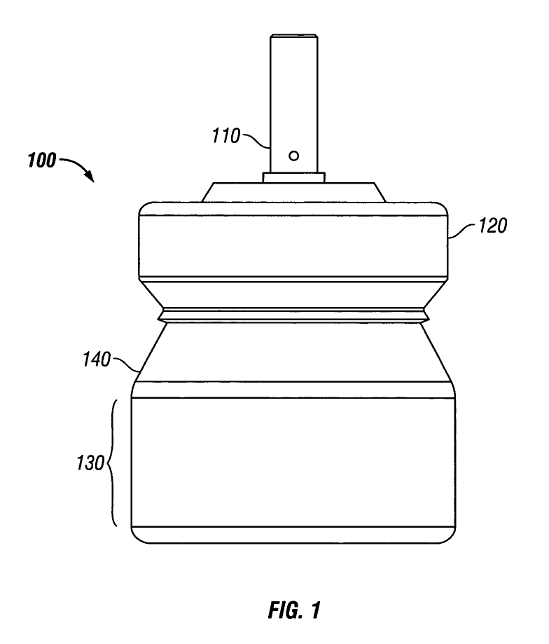

An exemplary primeless valve and minimal volume canister Assembly 100 of the invention is depicted in FIG. 1. Assembly 100 is made up of Canister 140, Cap 120 and Valve 110. Note that length of Body Wall 130 of the Canister 140 may be further reduced to decrease the internal volume of Canister 140 and minimize the required ullage Ullage is a term known to those skilled in the art and represents the minimum residual volume level from which the metering chamber of the valve can effectively be filled with drug formulation. If there is less than this ullage (minimum volume), erratic dose metering from the valve may be experienced and consistent delivery may be no longer possible. In one variation, Valve 110 is configured to deliver a unit dose of active agent upon the first and any subsequent actuation of the valve stem

The minimal volume canisters of the invention may be of any dimension and geometry. They are configured to minimize the amount of the active agent within the canister in order to avoid potential toxicity, overdose, or abuse of the agent contained therein. The volume of the canister is reduced by reducing the dimensions of the canister itself, not by placing an insert or second canister within it. The canister may be used with any suitable inhaler. The containers described here generally include a pressurized canister, a valve fixedly attached to the proximal end of the canister, and a metering chamber.

The pressurized canister may hold any active agent. For example, the active agent may be a central nervous system (CNS) agent such as ondansetron, granisetron, ropinirole, sildenafil, a benzodiazepine, a barbiturate, an ergot alkyloid, such as dihydroergotamine, a narcotic analgesic such as an opioid, including fentanyl, morphine, hydromorphone, oxycodone, or a metabolic replacement or modulation agent, such as PTH, insulin, GLP-1, PPT, exenatide, or neuropeptides such as PYY. Combinations, salts, analogs, and derivative of the aforementioned active agents are also contemplated.

Typical CNS therapies would require only about 2 to about 10 actuations to provide a single treatment. Examples could include fentanyl or other opiates, for breakthrough pain, dihydroergotamine or sumatriptan for migraine treatment, or Copaxone® for MS treatment, sedatives for insomnia, or PTH for osteoporosis. Assuming a maximum metering volume of 200 microliters for 10 actuations, canisters with volumes less than about 6.0 ml, including headspace are desirable. In some variations, the total volume of the drug is less than or equal to about 75% of the total volume of the canister. In other variations, the total volume of the drug is less than or equal to about 50% of the total volume of the canister.

1. A pressurized metered dose inhaler comprising:
a pressurized canister of a medicament comprising a primeless valve wherein said pressurized canister has a total volume of less than about 10 mLs.

2. The pressurized metered dose inhaler as described in claim 1 wherein said pressurized canister has a ullage of less than about 0.9 mLs.

3. The pressurized metered dose inhaler as described in claim 1 wherein said pressurized canister contains about 20 or fewer discharge volumes of medicament.

4. The pressurized metered dose inhaler as described in claim 1 wherein said primeless valve delivers a discharge volume of about 10 microliters to about 200 microliters.

5. The pressurized metered dose inhaler as described in claim 1 wherein said pressurized canister has a total volume of less than about 6 ml.

6. The pressurized metered dose inhaler as described in claim 1 wherein said pressurized canister has a total volume of less than about 5 ml.

7. The pressurized metered dose inhaler of claim 1 wherein the medicament is a central nervous system agent.

8. The pressurized metered dose inhaler of claim 6 wherein the central nervous system medicament is selected from the group consisting of ondansetron, granisetron, ropinirole, sildenafil, a benzodiapine, a barbiturate, an ergot alkyloid, dihydroergotamine, a narcotic analgesic, an opioid, fentanyl, morphine, hydromorphone, oxicodone, a metabolic replacement or modulation agent, PTH (parathyroid hormone), insulin, GLP-1, PPT, exenatide, neuropeptides, PPY, and combinations, salts, analogs, and derivatives thereof.

9. The pressurized metered dose inhaler of claim 1 wherein the total volume of the medicament is less than or equal to 75% of the total volume of the canister.

10. The pressurized metered dose inhaler of claim 1 wherein the total volume of the medicament is less than or equal to 50% of the total volume of the canister.

11. The pressurized metered dose inhaler of claim 1 wherein the diameter of the pressurized canister is in the range of about 8 mm to about 30 mm.

12. The pressurized metered dose inhaler as described in claim 1 wherein said primeless valve will discharge the proper volume of medicament repetitively wherein the period of time between said repetitive discharges is in the range of about 1 day to about 2 years.
